(19) United States
(12) Patent Application Publication
(10) Pub. No.: US 2007/0021405 A1
(43) Pub. Date: Jan. 25, 2007

(54) ARYL- AND HETEROARYLPIPERIDINECARBOXYLATE-DERIVATIVES METHODS FOR THEIR PREPARATION AND USE THEREOF AS FATTY ACID AMIDO HYDROLASE ENZYME INHIBITORS

(75) Inventors: Ahmed ABOUABDELLAH, Thiais (FR); Antonio ALMARIO GARCIA, Chatenay Malabry (FR); Christian HOORNAERT, Antony (FR); Patrick LARDENOIS, Bourg-la-Reine (FR); Frank MARGUET, Verrieres le Buisson (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/465,238

(22) Filed: Aug. 17, 2006

Related U.S. Application Data
(63) Continuation of application No. PCT/FR05/00453, filed on Feb. 25, 2005.

(30) Foreign Application Priority Data
Feb. 26, 2004 (FR) 0401950

(51) Int. Cl. C07D 417/02; C07D 413/02; C07D 403/02

(52) U.S. Cl. 514/210.19; 544/236; 544/237; 544/238; 544/60; 544/110; 544/359; 544/209; 544/405; 544/333; 546/122; 544/284; 544/280

(57) ABSTRACT

The present invention comprises compounds corresponding to the general formula (I): 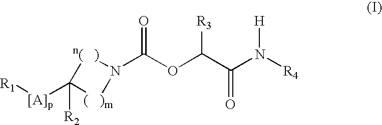
in which m, n=1 to 3 and m+n=2 to 5; p=1 to 7; A=single bond or X, Y and/or Z; X=optionally substituted methylene; Y=C₂-alkenylene, which is optionally substituted, or C₂-alkynylene; Z=C₃₋₇-cycloalkyl; R₁ represents a group of aryl or heteroaryl type; R₂ represents a hydrogen or fluorine atom or a hydroxyl, C₁₋₆-alkoxy or NR₈R₉ group; R₃ represents a hydrogen atom or a C₁₋₆-alkyl group; R₄ represents a hydrogen atom or a C₁₋₆-alkyl, C₃₋₇-cycloalkyl or C₃₋₇-cycloalkyl-C₁₋₃-alkyl group; in the base form or in the form of an addition salt with an acid, of a hydrate or of a solvate. The compounds are useful in the treatment of a number of diseases and/or pathological conditions such as chronic pain, dizziness, vomiting, nausea, eating disorders, neurological and psychiatric pathologies, acute or chronic neurodegenerative diseases, epilepsy, sleep disorders, cardiovascular diseases, renal ischaemia, cancers, disorders of the immune system, allergic diseases, parasitic, viral or bacterial infectious diseases, inflammatory diseases, osteoporosis, eye conditions, pulmonary conditions, gastrointestinal diseases or urinary incontinence.

### CROSS-REFERENCE TO RELATED APPLICATIONS

This application is a continuation of International Application No. PCT/FR2005/000453 filed on Feb. 25, 2005 which is incorporated herein by reference in its' entirety which also claims benefit of priority of French Patent Application No. 04/01950 filed on Feb. 26, 2004.

### FIELD OF THE INVENTION

The present invention relates generally to compounds and compositions for the treatment of neurological diseases that cause neurogenic and neuropathic pain, inflammatory diseases, renal ishaemia, cardiovascular disease and other pathologies caused by the presence of endogenous cannabinoids and or other substrates resulting from the metabolic activities of fatty acid amido hydrolase (FAAH).

### BACKGROUND OF THE INVENTION

Aryl- and heteroarylpiperidinecarboxylate derivatives are known to be useful in the treatment of numerous metabolic diseases in varying degrees. Methods for their preparation and their use in a number of therapeutic areas are also well known.

Phenylalkylcarbamate and dioxanyl-2-alkyl-carbamate derivatives and derivatives of aryloxyalkyl-carbamate are disclosed respectively in the documents FR 2 850 377 A, WO 2004/020430 A2 and PCT/FR2005/00028, as being are inhibitors of the enzyme Fatty Acid Amido Hydrolase (FAAH). These teachings and their disclosures are hereby incorporated by reference herein.

The enzyme fatty acid amido hydrolase (FAAH) (Chemistry and Physics of Lipids, (2000), 108, 107-121) catalyses the hydrolysis of endogenous derivatives of amides and esters of various fatty acids, such as N-arachidonoylethanolamine (anandamide), N-palmitoylethanolamine, N-oleoylethanolamine, oleamide or 2-arachidonoylglycerol. These derivatives have various pharmacological activities by interacting, inter alia, with the cannabinoid and vanilloid receptors.

The compounds of the present invention block this decomposition pathway and increase the tissue level of these endogenous substances. They can therefore be used in the prevention and treatment of pathologies in which endogenous cannabinoids and/or any other substrate metabolized by the enzyme FAAH are involved

These compounds are useful in the treatment of a wide variety of diseases, disorders and pathogenic conditions such as:
Pain, in particular acute or chronic neurogenic pain such as migraine headaches, neuropathic pain, including forms associated with the herpes virus and with diabetes; acute or chronic pain associated with inflammatory diseases such as arthritis, rheumatoid arthritis, osteoarthritis, spondylitis, gout, vasculitis, Crohn's disease, irritable bowel syndrome; acute or chronic peripheral pain; dizziness, vomiting, nausea, in particular nausea resulting from chemotherapy; eating disorders, in particular anorexia and cachexia of various natures; neurological and psychiatric pathologies, tremors, dyskinesias, dystonias, spasticity, obsessive-compulsive behaviour, Tourette's syndrome, all forms of depression and of anxiety of any nature and origin, mood disorders, psychoses; acute and chronic neurodegenerative diseases: Parkinson's disease, Alzheimer's disease, senile dementia, Huntington's chorea, lesions related to cerebral ischaernia and to cranial and medullary trauma;epilepsy;sleep disorders, including sleep apnoea; cardiovascular diseases, in particular hypertension, cardiac arrhythmias, arteriosclerosis, heart attack, cardiac ischaemia and renal ischaemia; cancers: benign skin tumours, brain tumours and papillomas, prostate tumours, cerebral tumours (glioblastomas, medulloepitheliomas, medulloblastomas, neuroblastomas, tumours of embryonic origin, astrocytomas, astroblastomas, ependymomas, oligodendrogliomas, plexus tumour, neuroepitheliomas, epiphyseal tumour, ependymoblastomas, malignant meningiomas, sarcomatosis, malignant melanomas, schwannomas); disorders of the immune system, in particular autoimmune diseases: psoriasis, lupus erythematosus, diseases of the connective tissue or collagen diseases, Sjögren's syndrome, ankylosing spondylitis, undifferentiated spondylitis, Behcet's disease, cancers: benign skin tumours, brain tumours and papillomas, prostate tumours, cerebral tumours (glioblastomas, medulloepitheliomas, medulloblastomas, neuroblastomas, tumours of embryonic origin, astrocytomas, astroblastomas, ependymomas, oligodendrogliomas, plexus tumour, neuroepitheliomas, epiphyseal tumour, ependymoblastomas, malignant meningiomas, sarcomatosis, malignant melanomas, schwannomas); disorders of the immune system, in particular autoimmune diseases: psoriasis, lupus erythematosus, diseases of the connective tissue or collagen diseases, Sjögren's syndrome, ankylosing spondylitis, undifferentiated spondylitis, Behcet's disease, autoimmune haemolytic anaemia, multiple sclerosis, amyotrophic lateral sclerosis, amyloidosis, graft rejection, diseases affecting the plasmocytic line; allergic diseases: immediate or delayed hypersensitivity, allergic rhinitis or conjunctivitis, contact dermatitis; parasitic, viral or bacterial infectious diseases: AIDS, meningitis; inflammatory diseases, in particular joint diseases: arthritis, rheumatoid arthritis, osteoarthritis, spondylitis, gout, vasculitis, Crohn's disease, irritable bowel syndrome; osteoporosis; eye conditions: ocular hypertension, glaucoma; pulmonary conditions: diseases of the respiratory tract, bronchospasm, coughing, asthma, chronic bronchitis, chronic obstruction of the respiratory tract gastrointestinal diseases: irritable bowel syndrome, inflammatory intestinal disorders, ulcers, diarrhoea;urinary incontinence and bladder inflammation.

Therefore, there still exists a need to find and develop products which are inhibitors of the enzyme FAAH as a means to treat and cure a patient afflicted with one of these conditions. The compounds of the present invention disclosed and claimed herein meet this aim.

### SUMMARY OF THE INVENTION

The present invention comprises compounds corresponding to the general formula (I): 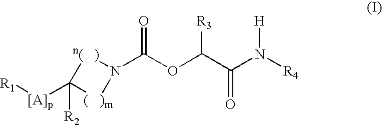
in which m, n=1 to 3 and m+n=2 to 5; p=1 to 7; A=single bond or X, Y and/or Z; X=optionally substituted methylene; Y═C₂-alkenylene, which is optionally substituted, or C₂-alkynylene; Z=C₃₋₇-cycloalkyl; R₁ represents a group of aryl or heteroaryl type; R₂ represents a hydrogen or fluorine atom or a hydroxyl, C₁₋₆-alkoxy or NR₈R₉ group; R₃ represents a hydrogen atom or a C₁₋₆-alkyl group; R₄ represents a hydrogen atom or a C₁₋₆-alkyl, C₃₋₇-cycloalkyl or C₃₋₇-cycloalkyl-C₁₋₃-alkyl group; in the base form or in the
form of an addition salt with an acid, a hydrate or a solvate. The compounds are useful in the treatment of a number of diseases and/or pathological conditions such as chronic pain, dizziness, vomiting, nausea, eating disorders, neurological and psychiatric pathologies, acute or chronic neurodegenerative diseases, epilepsy, sleep disorders, cardiovascular diseases, renal ischaemia, cancers, disorders of the immune system, allergic diseases, parasitic, viral or bacterial infectious diseases, inflammatory diseases, osteoporosis, eye conditions, pulmonary conditions, gastrointestinal diseases or urinary incontinence.

### DETAILED DESCRIPTION OF THE INVENTION

The compounds of the present invention correspond to the general formula (I): 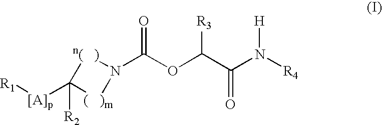
in which
m and n represent integers ranging from 1 to 3 such that m +n is an integer ranging from 2 to 5;
p represents an integer ranging from 1 to 7;
A represents a single bond or is chosen from one or more groups X, Y and/or Z;
X represents a methylene group optionally substituted by one or two C₁₋₆-alkyl, C₃₋₇-cycloalkyl or C₃₋₇-cycloalkyl-C₁₋₃-alkylene groups;
Y represents either a C₂-alkenylene group optionally substituted by one or two C₁₋₆-alkyl, C₃₋₇-cycloalkyl or C₃₋₇-cycloalkyl-C₁₋₃-alkylene groups; or a C₂-alkynylene group;
Z represents a group of formula: 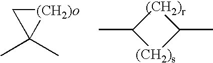
o represents an integer ranging from 1 to 5;
r and s represent integers and are defined such that r+s is a number ranging from 1 to 5;
R₁ represents an R₅ group optionally substituted by one or more R₆ and/or R₇ groups;
R₂ represents a hydrogen or fluorine atom or a hydroxyl, C₁₋₆-alkoxy or NR₈R₉ group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, pyridazinyl, pyrazinyl, triazinyl, pyrrolyl, furyl, thienyl, imidazolyl, oxazolyl, thiazolyl, pyrazolyl, isoxazolyl, isothiazolyl, oxadiazolyl, thiadiazolyl, triazolyl, tetrazolyl, naphthyl, quinolinyl, tetrahydroquinolinyl, isoquinolinyl, tetrahydroisoquinolinyl, quinazolinyl, quinoxalinyl, phthalazinyl, cinnolinyl, naphthyridinyl, benzofuranyl, dihydrobenzofuranyl, benzothienyl, dihydrobenzothienyl, indolyl, indolinyl, indazolyl, isoindolyl, benzimidazolyl, benzoxazolyl, benzisoxazolyl, benzothiazolyl, benzisothiazolyl, benzotriazolyl, benzoxadiazolyl, benzothiadiazolyl, pyrrolopyridyl, furopyridyl, thienopyridyl, imidazopyridyl, oxazolopyridyl, thiazolopyridyl, pyrazolopyridyl, isoxazolopyridyl or isothiazolopyridyl;
R₆ represents a halogen atom or a cyano, nitro, C₁₋₆-alkyl, C₃₋₇-cycloalkyl, C₁₋₆-alkoxy, hydroxyl, C₁₋₆-thioalkyl, C₁₋₆-fluoroalkyl, C₁₋₆-fluoroalkoxy, C₁₋₆-fluorothioalkyl, NR₈R₉, NR_{8COR}₉, NR₈CO₂R₉, NR₈SO₂R₉, COR₈, CO₂R₈, CONR₈R₉, SO₂R₈, SO₂NRSR₉ or —O—(C₁₋₃-alkylene)-O— group or a ring chosen from the azetidine, pyrrolidine, piperidine, morpholine, thiomorpholine, azepine or piperazine rings, this ring optionally being substituted by a C₁₋₆-alkyl or benzyl group;
R₇ represents a phenyl, phenyloxy, benzyloxy, naphthyl, pyridyl, pyiimidinyl, pyridazinyl or pyrazinyl group; it being possible for the R₇ group or groups to be substituted by one or more R₆ groups which are identical to or different from one another;
R₈ and R₉ represent, independently of one another, a hydrogen atom or a C₁₋₆-alkyl group.

In the context of the invention, the compounds of general formula (I) can thus comprise several groups A which are identical to or different from one another.

Among the compounds of general formula (I), a first subgroup of compounds is composed of the compounds for which:
m and n represent integers equal to 1 or 2 such that m+n is an integer ranging from 2 to 4;
p represents an integer ranging from 1 to 3;
A represents a single bond or a methylene or C₂-alkynylene group;
R₁ represents an R₅ group optionally substituted by one or more R₆ and/or R₇ groups;
R₂ represents a hydrogen atom or a hydroxyl group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, imidazolyl, thiazolyl, pyrazolyl, isoxazolyl, oxadiazolyl, naphthyl, tetrahydroquinolinyl, isoquinolinyl, tetrahydroisoquinolinyl, indolyl, indolinyl, benzimidazolyl, benzoxazolyl, benzothiazolyl, benzotriazolyl or pyrrolopyridyl;
R₆ represents a halogen atom, more particularly a bromine, a chlorine or a fluorine, or a cyano, C₁₋₆-alkyl, more particularly a methyl, a butyl or an isobutyl, C₃₋₇-cycloalkyl, more particularly a cyclopentyl, C₁₋₆-alkoxy, more particularly a methoxy or an ethoxy, or C₁₋₆-fluoroalkyl, more particularly a trifluoromethyl, group or a pyrrolidine or piperidine ring, this ring optionally being substituted by a C₁₋₆-alkyl group, more particularly an isopropyl;
R₇ represents a phenyl group which can be substituted by one or more R₆ groups which are identical to or different from one another.

Among the compounds of general formula (I), a second subgroup of compounds is composed of the compounds for which:
m and n represent integers equal to 1 or 2 such that m+n is an integer ranging from 2 to 4;
p represents an integer ranging from 1 to 3;
A represents a single bond or a methylene or C₂-alkynylene group;
R₁ represents an R₅ group optionally substituted by one or more R₆ and/or R₇ groups;
R₂ represents a hydrogen atom or a hydroxyl group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, thiazolyl, isoxazolyl, naphthyl or isoquinolinyl;
R₆ represents a halogen atom, more particularly a bromine, a chlorine or a fluorine, or a cyano, C₁₋₆-alkyl, more particularly a methyl, a butyl or an isobutyl, C₃₋₇-cycloalkyl, more particularly a cyclopentyl, C₁₋₆-alkoxy, more particularly a methoxy or an ethoxy, or C₁₋₆-fluoroalkyl, more particularly a trifluoromethyl, group or a pyrrolidine or piperidine ring, this ring optionally being substituted by a C₁₋₆-alkyl group, more particularly an isopropyl;
R₇ represents a phenyl group which can be substituted by one or more R₆ groups which are identical to or different from one another.

Among the compounds of general formula (I), a third subgroup of compounds is composed of the compounds for which:
m, n, p, A and R₁ are as defined in the first subgroup defined above;
R₃ represents a hydrogen atom;
R₄ represents a hydrogen atom or a C₁₋₆-alkyl group, more particularly a methyl.

Among the compounds of the subgroups defined above, the following compounds are particularly preferred:
2-(methylamino)-2-oxoethyl 4-{5-[4-(trifluoro-methyl)phenyl]pyrid-2-yl}piperidine-1-carboxylate
2-(methylamino)-2-oxoethyl 4-(4′-chlorobiphenyl-4-yl)-4-hydroxypiperidine-1-carboxylate
2-(methylamino)-2-oxoethyl 4-(4′-ethoxybiphenyl-4-yl)-4-hydroxypiperidine-1-carboxylate
2-(methylamino)-2-oxoethyl 4-(3′,4′-dichlorobiphenyl-4-yl)-4-hydroxypiperidine-1-carboxylate
2-(methylamino)-2-oxoethyl 4-(3′-chloro-4′-fluorobiphenyl-4-yl)-4-hydroxypiperidine-1-carboxylate
2-(methylamino)-2-oxoethyl 4-[(6-cyclopentylpyrid-2-yl)methyl]piperidine-1-carboxylate
2-(methylamino)-2-oxoethyl 4-[2-(3-chloro-phenyl)ethyl]piperidine-1-carboxylate
2-(methylamino)-2-oxoethyl 4-[2-(4-chloro-phenyl)ethyl]piperidine-1-carboxylate
2-(methylamino)-2-oxoethyl 4-{2-[3-(trifluoro-methyl)phenyl]ethyl }piperidine-1-carboxylate
2-(methylamino)-2-oxoethyl 4-{2-[4-(trifluoro-methyl)phenyl]ethyl}piperidine-1-carboxylate
2-(methylamino)-2-oxoethyl 4-(2-(biphenyl-3-yl)ethyl)-piperidine-1-carboxylate
2-(methylamino)-2-oxoethyl 4-[2-(1-naphthyl)-ethyl]piperidine-1-carboxylate
2-(methylamino)-2-oxoethyl 4-[2-(2-naphthyl)-ethyl]piperidine-1-carboxylate
2-(methylamino)-2-oxoethyl 4-[2-(6-cyclopentylpyiid-2-yl)ethyl]piperidine-1-carboxylate
2-(methylamino)-2-oxoethyl 4-[2-(6-(pyrrolidin-1-yl)pyrid-2-yl)ethyl]piperidine-1-carboxylate
2-(methylamino)-2-oxoethyl 4-(2-(isoquinolin-1-yl)ethyl)piperidine-1-carboxylate
2-(methylamino)-2-oxoethyl 4-[3-(3-chloro-phenyl)propyl]piperidine-1-carboxylate
2-(methylamino)-2-oxoethyl 4-[3-(4-chloro-phenyl)propyl]piperidine-1-carboxylate
2-(methylamino)-2-oxoethyl 4-{3-[3-(trifluoro-methyl)phenyl]propyl}piperidine-1-carboxylate
2-(methylamino)-2-oxoethyl 4-{3-[4-(trifluoro-methyl)phenyl]propyl}piperidine-1-carboxylate
2-(methylamino)-2-oxoethyl 4-[3-(3-cyano-phenyl)propyl]piperidine-1-carboxylate
2-(methylamino)-2-oxoethyl 4-(3-(biphenyl-2-yl)propyl)piperidine-1-carboxylate
2-(methylamino)-2-oxoethyl 4-(3-(biphenyl-3-yl)propyl)piperidine-1-carboxylate
2-(methylamino)-2-oxoethyl 4-[3-(1-naphthyl)-propyl]piperidine-1-carboxylate
2-(methylamino)-2-oxoethyl 4-[3-(2-naphthyl)-propyl]piperidine-1-carboxylate
2-(methylamino)-2-oxoethyl 4-[3-(1,3-thiazol-2-yl)propyl]piperidine-1-carboxylate
2-(methylamino)-2-oxoethyl 4-[(3-chloro-phenyl)ethynyl]piperidine-1-carboxylate
2-(methylamino)-2-oxoethyl 4-[(4-chloro-phenyl)ethynyl]piperidine-1-carboxylate
2-(methylamino)-2-oxoethyl 4-(biphenyl-3-ylethynyl)piperidine-1-carboxylate
2-(methylamino)-2-oxoethyl 4-(1-naphthylethynyl)-piperidine-1-carboxylate
2-(methylamino)-2-oxoethyl 4-(2-naphthylethynyl)-piperidine-1-carboxylate
2-(methylamino)-2-oxoethyl 4-(3-(biphenyl-2-yl)prop-2-yn-1-yl)piperidine-1-carboxylate
2-(methylamino)-2-oxoethyl 4-[(6-(pyrrolidin-1-yl)pyrid-2-yl)methyl]piperidine-1-carboxylate

Among the compounds of general formula (I), one subfamily of compounds is composed of the compounds corresponding to the general formula (I′): 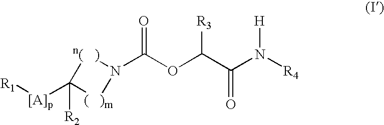
in which
m and n represent integers ranging from 1 to 3 such that m+n is an integer ranging from 2 to 5;
p represents an integer ranging from 1 to 7;
A represents a single bond or is chosen from one or more groups X, Y and/or Z;
X represents a methylene group optionally substituted by one or two C₁₋₆-alkyl, C₃₋₇-cycloalkyl or C₃₋₇-cycloalkyl-C₁₋₃-alkylene groups;
Y represents either a C₂-alkenylene group optionally substituted by one or two C₁₋₆-alkyl, C₃₋₇-cycloalkyl or C₃₋₇-cycloalkyl-C₁₋₃-alkylene groups; or a C₂-alkynylene group;
Z represents a group of formula: 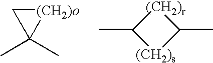
o represents an integer ranging from 1 to 5;
r and s represent integers and are defined such that r+s is a number ranging from 1 to 5;
R₁ represents an R₅ group optionally substituted by one or more R₆ and/or R₇ groups;
R₂ represents a hydrogen or fluorine atom or a hydroxyl, C₁₋₆-alkoxy or NR₈R₉ group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, pyridazinyl, pyrazinyl, triazinyl, pyrrolyl, furyl, thienyl, imidazolyl, oxazolyl, thiazolyl, pyrazolyl, isoxazolyl, isothiazolyl, oxadiazolyl, thiadiazolyl, triazolyl, tetrazolyl, naphthyl, quinolinyl, tetrahydroquinolinyl, isoquinolinyl, tetrahydroisoquinolinyl, quinazolinyl, quinoxalinyl, phthalazinyl, cinnolinyl, naphthyridinyl, benzofuranyl, dihydrobenzofuranyl, benzothienyl, dihydrobenzothienyl, indolyl, indolinyl, indazolyl, isoindolyl, benzimidazolyl, benzoxazolyl, benzisoxazolyl, benzothiazolyl, benzisothiazolyl, benzotriazolyl, benzoxadiazolyl, benzothiadiazolyl, pyrrolopyridyl, furopyridyl, thienopyridyl, imidazopyridyl, oxazolopyridyl, thiazolopyridyl, pyrazolopyridyl, isoxazolopyridyl or isothiazolopyridyl;
R₆ represents a halogen atom or a cyano, nitro, C₁₋₆-alkyl, C₁₋₆-alkoxy, hydroxyl, C₁₋₆-thioalkyl, C₁₋₆-fluoroalkyl, C₁₋₆-fluoroalkoxy, C₁₋₆-fluorothioalkyl, NR₈R₉, NR₈COR₉, NR₈CO₂R₉, NR₈SO₂R₉, COR₈, CO₂R₈, CONR₈R₉, SO₂R₈, SO₂NR₈R₉ or —O—(C₁₋₃-alkylene)-O— group;
R₇ represents a phenyl, phenyloxy, benzyloxy, naphthyl, pyridyl, pyrimidinyl, pyridazinyl or pyrazinyl group; it being possible for the R₇ group or groups to be substituted by one or more R₆ groups which are identical to or different from one another;
R₈ and R₉ represent, independently of one another, a hydrogen atom or a C₁₋₆-alkyl group or form, with the atom or atoms which carry them, a ring chosen from an azetidine, pyrrolidine, piperidine, morpholine, thiomorpholine, azepine or piperazine ring optionally substituted by a C₁₋₆-alkyl or benzyl group.

Among the compounds of general formula (I′), a first subgroup of compounds is composed of the compounds for which:
m and n represent integers equal to 1 or 2 such that m+n is an integer ranging from 2 to 4;
p represents an integer equal to 1 or 2;
A represents a single bond or a methylene group;
R₁ represents an R₅ group optionally substituted by one or more R₆ and/or R₇ groups;
R₂ represents a hydrogen or fluorine atom or a hydroxyl, C₁₋₆-alkoxy or NR₈R₉ group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, imidazolyl, naphthyl, tetrahydroquinolinyl, tetrahydroiso-quinolinyl, indolyl, indolinyl, benzimidazolyl, benzotriazolyl or pyrrolopyridyl;
R₆ represents a halogen atom, more particularly a bromine, a chlorine or a fluorine, or a C₁₋₆-alkyl, more particularly a methyl or a butyl, C₁₋₆-alkoxy, more particularly a methoxy or an ethoxy, or C₁₋₆-fluoroalkyl, more particularly a trifluoromethyl, group;
R₇ represents a phenyl group which can be substituted by one or more R₆ groups which are identical to or different from one another.

Among the compounds of general formula (I′), a second subgroup of compounds is composed of the compounds for which:
m, n, p, A and R₁ are as defined in the first subgroup defined above;
R₃ represents a hydrogen atom;
R₄ represents a hydrogen atom or a C₁₋₆-alkyl group, more particularly a methyl.

Mention may be made, among the compounds of general formula (I′), of the following compounds:
2-amino-2-oxoethyl 4-phenylpiperidine-1-carboxylate;
2-(methylamino)-2-oxoethyl 4-phenylpiperidine-1-carboxylate;
2-amino-2-oxoethyl 4-[3-(trifluoromethyl)-phenyl]piperidine-1-carboxylate;
2-(methylamino)-2-oxoethyl 4-[3-(trifluoro-methyl)phenyl]piperidine-1-carboxylate;
2-(methylamino)-2-oxoethyl 4-(4-phenyl-1H-imidazol-1-yl)piperidine-1-carboxylate;
2-(methylamino)-2-oxoethyl 4-(1H-1,2,3-benzotriazol-1-yl)piperidine-1-carboxylate;
2-(methylamino)-2-oxoethyl 4-(4-bromophenyl)-4-hydroxypiperidine-1-carboxylate;
2-(methylamino)-2-oxoethyl 4-(4′-fluorobiphenyl-4-yl)-4-hydroxypiperidine-1-carboxylate;
2-(methylamino)-2-oxoethyl 4-(4′-chlorobiphenyl-4-yl)-4-hydroxypiperidine-1-carboxylate;
2-(methylamino)-2-oxoethyl 4-hydroxy-4-(4′-methylbiphenyl-4-yl)piperidine-1-carboxylate;
2-(methylamino)-2-oxoethyl 4-(4′-butylbiphenyl-4-yl)-4-hydroxypiperidine-1-carboxylate;
2-(methylamino)-2-oxoethyl 4-hydroxy-4-[4′-(trifluoromethyl)biphenyl-4-yl]piperidine-1-carboxylate;
2-(methylamino)-2-oxoethyl 4-hydroxy-4-[4′-(methyloxy)biphenyl-4-yl]piperidine-1-carboxylate;
2-(methylamino)-2-oxoethyl 4-[4′-(ethyloxy)biphenyl-4-yl]-4-hydroxypiperidine-1-carboxylate;
2-(methylamino)-2-oxoethyl 4-(3′,4′-dichlorobiphenyl-4-yl)-4-hydroxypiperidine-1-carboxylate;
2-(methylamino)-2-oxoethyl 4-[3′-fluoro-4′-(methyloxy)biphenyl-4-yl]-4-hydroxypiperidine-1-carboxylate;
2-(methylamino)-2-oxoethyl 4-(3′-chloro-4′-fluoro-biphenyl-4-yl)-4-hydroxypiperidine-1-carboxylate;
2-(methylamino)-2-oxoethyl 4-(naphth-2-ylmethyl)-piperidine-1-carboxylate;
2-(methylamino)-2-oxoethyl 4-(biphenyl-4-ylmethyl)piperidine-1-carboxylate;
2-amino-2-oxoethyl 4-(1H-indol-1-ylmethyl)piperidine-1-carboxylate;
2-amino-2-oxoethyl 4-(2,3-dihydro-1H-indol-1-ylmethyl)piperidine-1-carboxylate;
2-amino-2-oxoethyl 4-(3,4-dihydroquinolin-1(2H)-ylmethyl)piperidine-1-carboxylate;
2-amino-2-oxoethyl 4-(3,4-dihydroisoquinolin-2( 1H)-ylmethyl)piperidine-1-carboxylate;
2-amino-2-oxoethyle 4-(1H-pyrrolo[2,3-b]pyrid-1-ylmethyl)piperidine-1-carboxylate;
2-amino-2-oxoethyl 4-(1H-benzimidazol-1-ylmethyl)piperidine-1-carboxylate;
2-amino-2-oxoethyl 4-[(4-phenyl-1H-imidazol-1-yl)methyl]piperidine-1-carboxylate;
2-amino-2-oxoethyl 3-(2-phenylethyl)pyrrolidine-1-carboxylate;
2-amino-2-oxoethyl 4-[2-(3,4-dihydroquinolin-1 (2H)-yl)ethyl]piperidine-1-carboxylate;
2-amino-2-oxoethyl 4-[2-(3,4-dihydroisoquinolin-2(1H)-yl)ethyl]piperidine-1-carboxylate;
2-amino-2-oxoethyl 4-[2-(1H-indol-1-yl)ethyl]-piperidine-1-carboxylate;
2-amino-2-oxoethyl 4-[2-(2,3-dihydro-1H-indol-1-yl)ethyl]piperidine-1-carboxylate;
2-amino-2-oxoethyl 4-[2-(1H-pyrrolo[2,3-b]pyrid-1-yl)ethyl]piperidine-1-carboxylate;
2-amino-2-oxoethyl 4-[2-(1H-benzimidazol-1-yl)ethyl]piperidine-1-carboxylate;
2-amino-2-oxoethyl 4-[2-(4-phenyl-1H-imidazol-1-yl)ethyl]piperidine-1-carboxylate.

The compounds of general formula (I) can comprise one or more asymmetric carbons. They can exist in the form of enantiomers or of diastereoisomers. These enantiomers and diastereoisomers, and their mixtures, including the racemic mixtures, form part of the invention.

The compounds of formula (I) can exist in the form of bases or of addition salts with acids. Such addition salts form part of the invention.

These salts are advantageously prepared with pharmaceutically acceptable acids but the salts of other acids, of use, for example, in the purification or the isolation of the compounds of formula (I), also form part of the invention. The compounds of general formula (I) can exist in the form of hydrates or of solvates, namely in the form of combinations or of associations with one or more molecules of water or with a solvent. Such hydrates and solvates also form part of the invention.

In the context of the invention:
C_{t-z}, where t and z can take the values from 1 to 7, is understood to mean a carbon chain which can have from t to z carbon atoms, for example C₁₋₃ a carbon chain which can have from 1 to 3 carbon atoms;
alkyl is understood to mean a saturated, linear or branched, aliphatic group; for example a C₁₋₆-alkyl group represents a linear or branched carbon chain of 1 to 6 carbon atoms, more particularly a methyl, ethyl, propyl, isopropyl, butyl, isobutyl, tert-butyl, pentyl or hexyl;
alkylene is understood to mean a saturated, linear or branched, divalent alkyl group, for example a C₁₋₃-alkylene group represents a linear or branched divalent carbon chain of 1 to 3 carbon atoms, more particularly a methylene, ethylene, 1-methylethylene or propylene;
cycloalkyl is understood to mean a cyclic alkyl group, for example a C₃₋₇-cycloalkyl group represents a cyclic carbon group of 3 to 7 carbon atoms, more particularly a cyclopropyl, cyclobutyl, cyclopentyl, cyclohexyl or cycloheptyl;
alkenylene is understood to mean an unsaturated divalent aliphatic group comprising 2 carbons, more particularly an ethylene;
C₂-alkynylene is understood to mean a —C≡C— group;
alkoxy is understood to mean an —O-alkyl group comprising a saturated, linear or branched, aliphatic chain;
thioalkyl is understood to mean an —S-alkyl group comprising a saturated, linear or branched, aliphatic chain;
fluoroalkyl is understood to mean an alkyl group, one or more hydrogen atoms of which have been substituted by a fluorine atom;
fluoroalkoxy is understood to mean an alkoxy group, one or more hydrogen atoms of which have been substituted by a fluorine atom;
fluorothioalkyl is understood to mean a thioalkyl group, one or more hydrogen atoms of which have been substituted by a fluorine atom;
halogen atom is understood to mean a fluorine, a chlorine, a bromine or an iodine.

The compounds of the invention can be prepared according to the method illustrated by the following scheme.

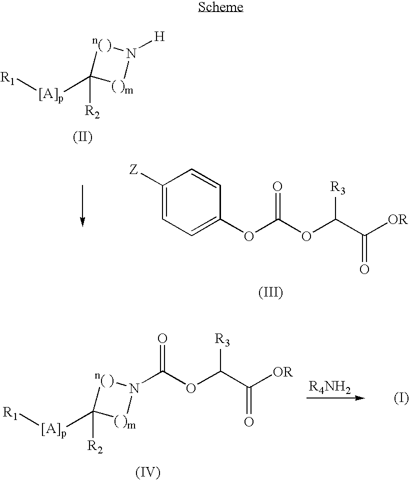

The compounds of the invention can be prepared by reacting an amine of general formula (II), in which R₁, A, R₂, p, m and n are as defined in the general formula (II), with a carbonate of general formula (III), in which Z represents a hydrogen atom or a nitro group, R₃ is as defined in the general formula (I) and R represents a methyl or ethyl group, in a solvent, such as toluene, dichloroethane, acetonitrile or a mixture of these solvents, at a temperature of between 0° C. and 80° C. The carbamate-esters of general formula (IV) thus obtained are subsequently converted to compounds of general formula (I) by aminolysis using an amine of general formula R₄NH₂, where R₄ is as defined in the general formula (I). The aminolysis reaction can be carried out in a solvent, such as methanol or ethanol, or a mixture of solvents, such as methanol and tetrahydrofuran.

The compounds of general formula (I) or (IV) in which R₁ represents a group of aryl-aryl, aryl-heteroaryl, heteroaryl-aryl or heteroaryl-heteroaryl type can also be prepared by reaction of the corresponding compounds of general formula (I) or (IV) for which R₅ is substituted by a chlorine, bromine or iodine atom or by a triflate group in the position where the R₇ group has to be introduced with an aryl- or heteroarylboronic acid derivative according to the Suziki reaction conditions (Chem. Rev., 1995, 95, 2457-2483) or with an aryl- or heteroaryltrialkylstannane derivative according to the Stille reaction conditions (Angew. Chem. Int. Ed., 1986, 25, 504-524).

The carbonates of general formula (III) can be prepared according to any method described in the literature, for example by reaction of an alcohol of general formula HOCHR₃COOR, where R represents a methyl or ethyl group, with phenyl or 4-nitrophenyl chloro-formate in the presence of a base, such as triethylamine or diisopropylethylamine.

The compounds of general formula (II) and the amines of general formula R₄NH₂, when their method of preparation is not described, are commercially available or are described in the literature or can be prepared according to various methods described in the literature or known to a person skilled in the art.

The compounds of general formula (IV) in which R₁, A, R₂, R₃, p, m and n are as defined in the general formula (I) and R represents a methyl or ethyl group are novel and also form part of the invention. They are of use as synthetic intermediates in the preparation of the compounds of general formula (I).

The examples which will follow illustrate the preparation of a few compounds of the invention. Whereas these examples are offered to best describe how to make and use the compounds of the present invention, these examples are not limiting and only serve to illustrate a number of specific embodiments of the present the invention. They are not to be construed as limiting the spirit and scope of the invention as defined by the claims that follow.

In the following examples, the microanalyses, the IR and NMR spectra and/or the LC-MS (Liquid Chromatography coupled to Mass Spectroscopy) confirm the structures and the purities of the compounds obtained.

M.p. (° C) represents the melting point in degrees Celsius.

The numbers shown in brackets in the titles of the examples correspond to those in the 1st column in the table below. The IUPAC nomenclature was used to name the compounds in the following examples. For example, for the biphenyl group, the following notation was observed:

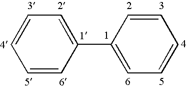

### EXAMPLE 1

### Compound No. 14

### 2-(Methylamino)-2-oxoethyl 4-(4-phenyl-1H-imidazol-1-yl)piperidine-1-carboxylate

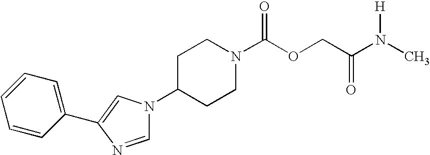

### 1. 1. 1,1-Dimethylethyl 4-[(methylsulphonyl)oxy]-piperidine-1-carboxylate

1.4 ml (17.9 mmol) of methanesulphonyl chloride are added dropwise with stirring to a solution, cooled with an ice bath, of 3.0 g (14.9 mmol) of 1,1-dimethylethyl 4-hydroxypiperidine-1-carboxylate and of 2.2 ml (17.9 mmol) of triethylamine in 60 ml of dichloromethane. Stirring is continued at 0° C. for one hour and then at ambient temperature for 4 hours. The reaction mixture is diluted with 100 ml of dichloromethane and is washed successively with 100 ml of an aqueous sodium hydrogencarbonate solution, with a saturated aqueous ammonium chloride solution and then with a saturated aqueous sodium chloride solution. The organic phase is dried over sodium sulphate and evaporated to dryness. The residue is subsequently triturated from a 50/50 mixture of cyclohexane and of diethyl ether to produce 3.7 g of product in the form of a white solid.

### 1.2. 1,1-Dimethylethyl 4-(4-phenyl-1H-imidazol-1-yl)piperidine-1-carboxylate

A solution of 4.0 g (27.9 mmol) of 4-phenyl-imidazole in 40 ml of N,N-dimethylformamide is added dropwise to a suspension, cooled with an ice bath, of 1.1 g (27.9 mmol) of sodium hydride (60% suspension in oil) in 30 ml of N,N-dimethylformamide. The mixture is subsequently stirred at ambient temperature for one hour, is then cooled to 0° C. and 2.6 g (9.3 mmol) of 1,1-dimethylethyl 4-[(methylsulphonyl)oxy]piperidine-1-carboxylate, obtained in stage 1.1., in solution in 20 ml of N,N-dimethylformamide, are added dropwise. The reaction mixture is subsequently heated at 80° C. for 2 hours. It is cooled to ambient temperature and diluted with 150 ml of water and 150 ml of ethyl acetate. Separation by settling is carried out and the aqueous phase is extracted twice with 100 ml of ethyl acetate. The organic phases are washed with two times 100 ml of water and then with 100 ml of a saturated aqueous sodium chloride solution. They are dried over sodium sulphate and evaporated to dryness. The residue is purified by chromatography on silica gel, elution being carried out with a 98/2 mixture of dichloromethane and of methanol, to produce 1.0 g of product in the form of a yellow oil.

### 1.3. 4-(4-Phenyl-1H-imidazol-1-yl)piperidine

5.6 ml (76.3 mmol) of trifluoroacetic acid are added dropwise to a solution, cooled with an ice bath, of 1.0 g (3.05 mmol) of 1,1-dimethylethyl 4-(4-phenyl-1H-imidazol-1-yl)piperidine-1-carboxylate, obtained in stage 1.2., in 60 ml of dichloromethane. The mixture is subsequently stirred at ambient temperature for one hour and is evaporated to dryness. The residue is taken up in 25 ml of water, and 2 ml of a 30% aqueous sodium hydroxide solution are added. The mixture is stirred for 30 minutes and is then extracted four times with 80 ml of dichloromethane. The organic phases are subsequently washed with a saturated aqueous sodium chloride solution, dried over sodium sulphate and evaporated to dryness to produce 0.7 g of product in the form of a yellow oil used as is in the following stage.

### 1.4. 2-(Ethyloxy)-2-oxoethyl 4-(4-phenyl-1H-imidazol-1-yl)piperidine-1-carboxylate

A solution of 1.0 g (4.4 mmol) of 4-(4-phenyl-1H-imidazol-1-yl)piperidine, prepared according to stage 1.3., and of 1.18 g (5.2 mmol) of ethyl [(phenyloxycarbonyl)oxy]acetate (J. Med. Chem., 1999, 42, 277-290) in 50 ml of toluene is heated at 60° C. overnight. The mixture is subsequently evaporated to dryness and the residue is taken up in 80 ml of ethyl acetate and 80 ml of water. Separation by settling is carried out and the aqueous phase is extracted with three times 80 ml of ethyl acetate. The organic phases are subsequently washed with 80 ml of a saturated aqueous sodium chloride solution. They are dried over sodium sulphate and evaporated to dryness. The residue is purified by chromatography on silica gel, elution being carried out with a 98/2 mixture of dichloromethane and of methanol, to produce 0.35 g of product.

### 1.5. 2-(Methylamino)-2-oxoethyl 4-(4-phenyl-1H-imidazol-1-yl)piperidine-1-carboxylate

0.35 g (0.98 mmol) of 2-(ethyloxy)-2-oxoethyl 4-(4-phenyl-1H-imidazol-1-yl)piperidine-1-carboxylate, obtained in stage 1.4., is dissolved in 7 ml of methanol. 1.5 ml (3 mmol) of a 2M solution of methylamine in tetrahydrofuran are added. After 16 hours at ambient temperature, a further 1 ml (2 mmol) of a 2M solution of methylamine in tetrahydrofuran is added and reaction is allowed to take place for an additional 6 hours. The mixture is evaporated to dryness and the residue is purified by chromatography on silica gel, elution being carried out with a 98/2 then 97/3, 96/4 and 95/5 mixture of dichloromethane and of methanol. Trituration is subsequently carried out from diethyl ether to produce 0.20 g of product in the form of a white solid.

Melting point (° C.): 192-194

LC-MS: M+H=343

¹H NMR (CDCl₃) δ (ppm): 7.75 (d, 2H), 7.60 (s, 1H), 7.40 (m, 2H), 7.25 (m, 2H), 6.05 (broad s, 1H), 4.65 (s, 2H), 4.35 (m, 2H), 4.15 (m, 1H), 3.05 (m, 2H), 2.90 (d, 3H), 2.20 (m, 2H), 2.05-1.85 (m, 2H).

### EXAMPLE 2

### Compound No. 32

### 2-(Methylamino)-2-oxoethyl 4-(4-bromophenyl)-4-hydroxypiperidine-1-carboxylate

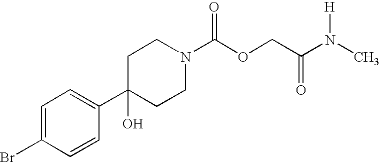

### 2.1. 2-(Ethyloxy)-2-oxyethyl 4-(4-bromophenyl)-4-hydroxypiperidine-1-carboxylate

A mixture of 2.24 g (10 mmol) of ethyl [(phenyloxycarbonyl)oxy]acetate and 2.56 g (10 mmol) of 4-(4-bromophenyl)-4-piperidinol in solution in 40 ml of toluene is heated at 50° C. for 20 hours. The solution is evaporated to dryness on a water bath under reduced pressure. An oil is obtained and is used directly in the following stage.

### 2.2. 2-(Methylamino)-2-oxoethyl 4-(4-bromophenyl)-4-hydroxypiperidine 1-carboxylate

The 2-(ethyloxy)-2-oxyethyl 4-(4-bromophenyl)-4-hydroxy-piperidine-1-carboxylate obtained in stage 2.1. is stirred for 3 hours in a 33% solution of methylamine in methanol. The solution is concentrated on a water bath under reduced pressure. The residue is purified by chromatography on silica gel, elution being carried out with ethyl acetate. 2.6 g of product are obtained in the form of an oil which gradually solidifies.

Melting point (° C.): 57-60

LC-MS: M+H=371

¹H NMR (d₆-DMSO) δ (ppm): 7.55 (broad s, 1H), 7.50 (d, 2H), 7.40 (d, 2H), 5.20 (s, 1H), 4.40 (s, 2H), 3.80 (m, 2H), 3.20 (m, 2H), 2.60 (d, 3H), 1.90-1.50 (m, 4H).

### EXAMPLE 3

### Compound No. 40

### 2-(Methylamino)-2-oxoethyl 4-(3′,4′-dichlorobiphenyl-4-yl)-4-hydroxypiperidine-1-carboxylate

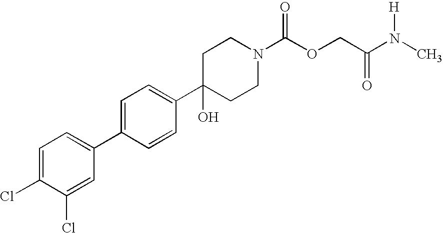

0.1 g (0.27 mmol) of 2-(methylamino)-2-oxoethyl 4-(4-bromophenyl)-4-hydroxypiperidine-1-carboxylate, obtained according to Example 2, 0.077 g (0.4 mmol) of 3,4-dichlorophenylboronic acid, 10 mg of tetrakis-(triphenylphosphine)palladium(0), 2 ml of 2M aqueous sodium carbonate solution, 0.5 ml of ethanol and 4 ml of toluene degassed beforehand with nitrogen are mixed. The mixture is heated at 80° C. with stirring for 20 hours. It is filtered under hot conditions through a hydrophobic cartridge, rinsing is carried out with tetrahydrofuran (THF) and evaporation to dryness is carried out. The residue is purified by LC-MS chromatography on a silica phase, elution being carried out with a cyclohexane/ethyl acetate/methanol gradient, to produce 0.069 g of crystalline product.

Melting point (° C.): 156-158

LC-MS: M+H=438

¹H NMR (d₆-DMSO) δ (ppm): 7.95 (s, 1H), 7.80 (m, 1H), 7.70 (m, 4H), 7.60 (m, 2H), 5.20 (s, 1H), 4.45 (s, 2H), 4.00 (m, 2H), 3.25 (m, 2H), 2.60 (d, 3H), 1.95 (m, 2H), 1.65 (m, 2H).

### EXAMPLE 4

### Compound No. 43

### 2-(Methylamino)-2-oxoethyl 4-(naphth-2-ylmethyl)piperidine-1-carboxylate

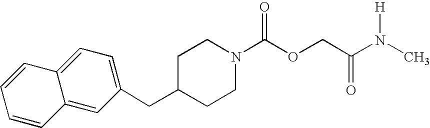

### 4.1. 1,1-Dimethylethyl 4-(naphth-2-ylmethyl)piperidine-1-carboxylate

8.0 ml of a 0.5N solution (4 mmol) of 9-borabicyclo[3.3.1]nonane in tetrahydrofuran are added under an argon atmosphere to a solution of 0.789 g (4 mmol) of 1,1-dimethylethyl 4-methylidenepiperidine-1-carboxylate (Tetrahedron Letters, 1996, 37(30), 5233-5234) in solution in 5 ml of tetrahydrofuran. The mixture is heated at reflux for 3 hours. It is cooled to ambient temperature and 0.787 g (3,8 mmol) of 2-bromonaphthalene in solution in 9 ml of N,N-dimethylformamide, 0.829 g (6.0 mmol) of potassium carbonate in solution in 1 ml of water and 0.16 g (0.20 mmol) of the [1,1′-bis(diphenylphosphino)ferrocene]dichloropalladium(II)-dichloromethane complex are added. The mixture is heated at reflux overnight. The reaction mixture is diluted with 150 ml of ethyl acetate and 50 ml of water. The organic phase is separated by settling and is washed with 25 ml of water and then with 25 ml of a saturated aqueous sodium chloride solution. It is dried over magnesium sulphate and evaporated under vacuum. The residue is purified by chromatography on silica gel, elution being carried out with a 99/1 then 95/5 and 90/10 mixture of cyclohexane and of ethyl acetate, to produce 0.79 g of product in the form of a colourless viscous liquid.

### 4.2. 4-(Naphth-2-ylmethyl)piperidine

0.79 g (2.43 mmol) of 1,1-dimethylethyl 4-(naphth-2-ylmethyl)piperidine-1-carboxylate, obtained in stage 4.1., is dissolved in 10 ml of dichloromethane, and 2 ml (25 mmol) of trifluoroacetic acid are added. The mixture is stirred at ambient temperature for 3 hours. It is evaporated under reduced pressure, then 4 ml of 1,2-dichloroethane are added and the mixture is again evaporated. The residue is taken up in a mixture of 50 ml of dichloromethane and of 15 ml of a 10% aqueous sodium hydroxide solution. The organic phase is separated by settling and the aqueous phase is extracted twice with 25 ml of dichloromethane. The organic phases are washed with 15 ml of a saturated aqueous sodium chloride solution, then dried over sodium sulphate and evaporated under vacuum to provide 0.52 g of product in the form of an orange oil used as is in the following stage.

### 4.3. 2-(Ethoxy)-2-oxoethyl 4-(naphth-2-ylmethyl)piperidine-1-carboxylate

A mixture of 0.52 g (2.3 mmol) of 4-(naphth-2-ylmethyl)piperidine, obtained in stage 4.2., and of 0.69 g (3.11 mmol) of ethyl [(phenyloxycarbonyl)oxy]acetate in 10 ml of toluene and 5 ml of acetonitrile is heated at 60° C. overnight. The mixture is evaporated under vacuum. The residue is purified by chromatography on silica gel, elution being carried out with a 90/10 then 85/15 and 80/20 mixture of cyclohexane and of ethyl acetate, to produce 0.56 g of product in the form of a colourless viscous liquid.

### 4.4. 2-(Methylamino)-2-oxoethyl 4-(naphth-2-ylmethyl)piperidine-1-carboxylate

0.54 g (1.52 mmol) of 2-(ethoxy)-2-oxoethyl 4-(naphth-2-ylmethyl)piperidine-1-carboxylate, obtained in stage 4.3., is dissolved in 3 ml of methanol, and 3 ml (6.0 mmol) of a 2M solution of methylamine in tetrahydrofuran are added. Reaction is allowed to take place overnight at ambient temperature, then 1.5 g of silica are added and the mixture is evaporated. The residue is purified by chromatography on silica gel, elution being carried out with a 98.5/1.5 and then 97/3 mixture of dichloromethane and of methanol. The product is subsequently recrystallized from a mixture of ethyl acetate and of diisopropyl ether to produce 0.43 g of product in the form of a white solid.

Melting point (° C.): 150-152

LC-MS: M+H 341

¹H NMR (CDCl₃) δ (ppm): 7.80 (m, 3H), 7.60 (s, 1H), 7.45 (m, 2H), 7.30 (d, 1H), 6.10 (m, 1H), 4.60 (s, 2H), 4.15 (m, 2H), 2.85 (d, 3H), 2.85-2.75 (m+d, 4H), 1.90-1.70 (m, 3H), 1.35-1.15 (m, 2H).

### EXAMPLE 5

### Compound No. 107

### 2-(Methylamino)-2-oxoethyl 4-[3-(4-chlorophenyl)prop-2-yn-1-yl]piperidine-1-carboxylate

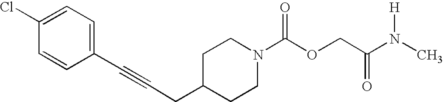

### 5.1. tert-Butyl 4-(2-oxoethyl)piperidine-1-carboxylate

70.9 g (167 mmol) of 1,1,1-tris(acetyloxy)-1,1-dihydro-1,2-benziodoxol-3-(1H)-one (Dess-Martin reagent) are added portionwise to a solution, cooled to 0° C., of 30.4 g (132 mmol) of tert-butyl 4-(2-hydroxyethyl)piperidine-1-carboxylate in 150 ml of dichloromethane. The mixture is stirred at ambient temperature for 2 hours, then 150 ml of a 10% aqueous sodium thiosulphate (Na₂S₂O₃) solution are added and stifling is continued for an additional 30 minutes. The organic phase is separated by settling, washed with a saturated aqueous sodium carbonate solution, dried over sodium sulphate and evaporated to dryness to produce 30.1 g (132 mmol) of product in the form of a colourless oil used as is in the following stage.

### 5.2. tert-Butyl 4-(3,3-dibromoprop-2-en-1-yl)piperidine-1-carboxylate

47.6 ml (531 mmol) of tribromomethane and then 59.6 g (531 mmol) of potassium tert-butoxide are added to a solution, cooled to −20° C., of 139.4 g (531 mmol) of triphenylphosphine in 440 ml of toluene. Stirring is continued at −20° C. for 15 minutes and then a solution of 30.1 g (131 mmol) of tert-butyl 4-(2-oxoethyl)piperidine-1-carboxylate, prepared in stage 5.1., in 240 ml of toluene is added. Stirring is continued at ambient temperature for 3 hours. 300 ml of diethyl ether are added, the solid formed is filtered off and the filtrate is evaporated. The residue is purified by chromatography on silica gel, elution being carried out with dichloromethane, to produce 32.6 g (85 mmol) of product in the form of a yellow oil.

### 5.3. tert-Butyl 4-(prop-2-yn-1-yl)piperidine-1-carboxylate

32.6 g (85 mmol) of tert-butyl 4-(3,3-dibromoprop-2-en-1-yl)piperidine-1-carboxylate, prepared in stage 5.2., are dissolved in 420 ml of anhydrous tetrahydrofuran. The solution is cooled to −78° C. and 106 ml of a 1.6M solution of n-butyllithium (170 mmol) in hexane, dissolved in 100 ml of anhydrous tetrahydrofuran, are added dropwise while stirring well. Stirring is continued at −78° C. for 3 hours and then at −20° C. for 1 hour. The mixture is cooled to −78° C. and 130 ml of a 1.25M solution of hydrochloric acid in ethanol are added. The mixture is subsequently reheated to ambient temperature over 1 hour. Water and ethyl acetate are added. The organic phase is separated by settling, washed with a saturated aqueous sodium chloride solution, dried over sodium sulphate and evaporated to dryness. The residue is purified by chromatography on silica gel, elution being carried out with dichloromethane and then with a 98/2 mixture of dichloromethane and of methanol, to produce 32.4 g (85.2 mmol) of product in the form of a colourless oil.

### 5.4. tert-Butyl 4-[3-(4-chlorophenyl)prop-2-yn-1-yl]piperidine-1-carboxylate

2.29 g (9.6 mmol) of 1-chloro-4-iodobenzene and 1.7 ml (12 mmol) of triethylamine are dissolved in 5 ml of tetrahydrofuran. 0.076 g (0.40 mmol) of cuprous iodide and 0.168 g (0.24 mmol) of the bis(triphenylphosphine)palladium dichloride complex are added under argon, followed, dropwise, by a solution of 1.78 g (8 mmol) of tert-butyl 4-(prop-2-yn-1-yl)piperidine-1-carboxylate, prepared in stage 5.3., in 3 ml of tetrahydrofuran. Stirring is continued overnight. 25 ml of water and 100 ml of ethyl acetate are added. The organic phase is separated by settling, washed successively with 25 ml of 10% aqueous ammonia, 25 ml of water and 25 ml of a saturated aqueous sodium chloride solution, dried over magnesium sulphate and evaporated to dryness. The residue is purified by chromatography on silica gel, elution being carried out with a 95/5 and then 90/10 mixture of cyclohexane and of ethyl acetate, to produce 2.15 g (6.44 mmol) of product in the form of a yellow oil.

### 5.5. 4-[3-(4-Chlorophenyl)prop-2-yn-1-yl]piperidine

2.13 g (6,38 mmol) of tert-butyl 4-[3-(4-chlorophenyl)prop-2-yn-1-yl]piperidine-1-carboxylate, obtained in stage 5.4., are dissolved in 15 ml of dichloromethane. A solution of 4.9 ml (63.8 mmol) of trifluoroacetic acid in 5 ml of dichloromethane is added dropwise. Reaction is allowed to take place at ambient temperature overnight and then the mixture is evaporated to dryness. 25 ml of dichloromethane are added and the mixture is again evaporated to dryness. The residue is subsequently taken up in a mixture of 70 ml of ethyl acetate, 10 ml of a 1N aqueous sodium hydroxide solution and 10 ml of 30% aqueous ammonia. The organic phase is separated by settling, washed with 2 times 10 ml of water and then with 10 ml of a saturated aqueous sodium chloride solution, dried over sodium sulphate and evaporated to dryness to produce 1.39 g (5.94 mmol) of product in the form of a brown oil used as is in the following stage.

### 5.6. 2-Ethoxy-2-oxoethyl 4-[3-(4-chlorophenyl)prop-2-yn-1-yl]piperidine-1-carboxylate

A solution of 1.39 g (5.94 mmol) of 4-[3-(4-chlorophenyl)prop-2-yn-1-yl]piperidine, prepared in stage 5.5, and of 1.86 g (8.33 mmol) of ethyl [(phenyloxycarbonyl)oxy]acetate in 12 ml of toluene is heated at 70° C. for 5 hours.

The mixture is evaporated to dryness and the residue is purified by chromatography on silica gel, elution being carried out with a 90/10 and then 80/20 mixture of cyclohexane and of ethyl acetate, to produce 1.89 g (5.19 mmol) of product in the form of a viscous oil.

### 5.7. 2-(Methylamino)-2-oxoethyl 4-[3-(4-chlorophenyl)prop-2-yn-1-yl]piperidine-1-carboxylate

0.91 g (2.51 mmol) of 2-ethoxy-2-oxoethyl 4-[3-(4-chlorophenyl)prop-2-yn-1-yl]piperidine-1-carboxylate, prepared in stage 5.6., is dissolved in 4 ml of methanol. 2.5 ml (25 mmol) of a 33% solution of methylamine in ethanol are added and the mixture is left overnight at ambient temperature. It is evaporated to dryness and the residue is purified by chromatography on silica gel, elution being carried out with a 99.5/0.5 and then 98/2 and 96/4 mixture of dichloromethane and of methanol. The product is crystallized from hexane and is then dried under vacuum to produce 0.50 g (1.43 mmol) of product in the form of a white powder.

Melting point (° C.): 101-103

LC-MS: M+H=349

¹H NMR (CDCl₃) δ (ppm): 7.20 (m, 4H), 6.30 (m, 1H), 4.50 (broad s, 2H), 4.10 (broad d, 2H), 2.75 (m+d, 5H), 2.30 (d, 2H), 1.85-1.60 (m, 3H), 1.35-1.15 (m, 2H),

### EXAMPLE 6

### Compound No. 83

### 2-(Methylamino)-2-oxoethyl 4-[3-(4-chlorophenyl)propyl]piperidine-1-carboxylate

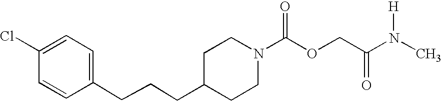

0.156 g (0.448 mmol) of 2-(methylamino)-2-oxoethyl 4-[3-(4-chlorophenyl)prop-2-yn-1-yl]piperidine-1-carboxylate, prepared according to Example 5, is dissolved in 2 ml of ethanol. 16 mg of platinum dioxide are added. The mixture is stirred under a hydrogen atmosphere at ambient pressure and ambient temperature for 2 hours and then at 40° C. for an additional 2 hours. The mixture is filtered through celite and the filtrate is evaporated. The residue is purified by HPLC chromatography on Nucleosil gel, elution being carried out with a 70/30/0 to 0/80/20 gradient of hexane, of ethyl acetate and of methanol, to produce 0.108 mg (0.306 mmol) of product in the form of a white solid.

Melting point (° C.): 118-120

LC-MS: M+H=353

¹H NMR (CDCl₃) δ (ppm): 7.25 (d, 2H), 7.10 (d, 2H), 6.05 (m, 1H), 4.60 (s, 2H), 4.10 (broad d, 2H), 2.90 (d, 3H), 2.80 (broad t, 2H), 2.60 (t, 2H), 1.75-1.55 (m, 4H), 1.45 (m, 1H), 1.35-1.05 (m, 4H).

### EXAMPLE 7

### Compound No. 74

### 2-(Methylamino)-2-oxoethyl 4-(2-(isoquinolin-1-yl)ethyl)-1-piperidinecarboxylate

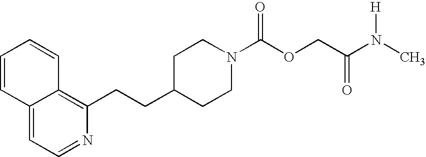

### 7.1. tert-Butyl 4-(iodomethyl)-1-piperidinecarboxylate

14.15 g (55.74 mmol) of iodine (12) are added in small portions to a solution, cooled to approximately 0° C., of 10 g (46.45 mmol) of tert-butyl 4-(hydroxymethyl)-1-piperidinecarboxylate, of 15.84 g (60.38 mmol) of triphenylphosphine and of 4.74 g (69.67 mmol) of imidazole in 200 ml of dichloromethane while keeping the temperature of the reaction medium between 0° C. and 5° C. Stirring is continued at 0° C. for 1 hour and then at ambient temperature for 4 hours.

100 ml of water and 300 ml of ethyl acetate are added. The organic phase is separated by settling, washed successively with a saturated aqueous sodium thiosulphate solution and a saturated aqueous sodium chloride solution, dried over sodium sulphate and concentrated under reduced pressure. The residue obtained is purified by chromatography on silica gel, elution being carried out with a 90/10 mixture of cyclohexane and of ethyl acetate. 13.70 g (42.13 mmol) of product are obtained in the form of a colourless oil.

### 7.2. tert-Butyl 4-(2-(isoquinolin-1-yl)ethyl)-1-piperidinecarboxylate

10 ml (20 mmol) of a solution (2M) of lithium diisopropylamide (LDA) in a mixture of tetrahydrofuran and of n-hexane are added dropwise to a solution, cooled to approximately −70° C., of 2.202 g (15.38 mmol) of 1-methyl-isoquinoline in 150 ml of tetrahydrofuran. Stirring is continued at −70° C. for 10 minutes and then a solution of 5 g (15.38 mmol) of tert-butyl 4-(iodomethyl)-1-piperidinecarboxylate, obtained in stage 7.1., in 30 ml of tetrahydrofuran is added slowly. After stirring at −70° C. for 30 minutes, 100 ml of a saturated aqueous ammonium chloride solution are added.

The mixture is allowed to return to ambient temperature and the aqueous phase is separated and then extracted 3 times with ethyl acetate. The combined organic phases are washed with a saturated aqueous sodium chloride solution, dried over sodium sulphate and concentrated under reduced pressure. The residue obtained is purified by chromatography on silica gel, elution being carried out with a 99/1 and then 98/2 mixture of dichloromethane and of methanol. 1.80 g (5.29 mmol) of product are obtained in the form of a yellow oil.

### 7.3. 1-(2-(Piperidin-4-yl)ethyl)isoquinoline

3.90 ml (23.50 mmol) of a solution of hydrochloric acid (6N) in isopropanol are added at ambient temperature to a solution of 1.60 g (4.70 mmol) of tert-butyl 4-(2-(isoquinolin-1-yl)ethyl)-1-piperidinecarboxylate, obtained in stage 7.2., in 15 ml of 1,4-dioxane. The reaction mixture is subsequently brought to approximately 60° C. for 12 hours.

The mixture is concentrated to dryness under reduced pressure. The hydrochloride obtained is taken up in 5 ml of water and then a 20% aqueous sodium hydroxide solution is slowly added with stirring to pH 9. The aqueous phase is extracted twice with chloroform and the combined organic phases are washed with a saturated aqueous sodium chloride solution, dried over sodium sulphate and concentrated under reduced pressure. 0.400 g (1.66 mmol) of product is obtained in the form of a brown oil.

### 7.4. 2-Ethoxy-2-oxoethyl 4-(2-(isoquinolin-1-yl)ethyl)-1-piperidinecarboxylate

A solution of 0.320 g (1.33 mmol) of 1-(2-(piperidin-4-yl)ethyl)isoquinoline, obtained in stage 7.3., and of 0.388 g (1.73 mmol) of ethyl [(phenyloxycarbonyl)oxy]acetate in 10 ml of toluene is heated at 70° C. for 18 hours.

The mixture is allowed to return to ambient temperature and concentrated under reduced pressure and then the residue thus obtained is purified by chromatography on silica gel, elution being carried out with a 40/60 mixture of ethyl acetate and of cyclohexane. 0.390 g (1.05 mmol) of product is thus obtained in the form of a viscous oil.

### 7.5. 2-(Methylamino)-2-oxoethyl 4-(2-(isoquinolin-1-yl)ethyl)-1-piperidinecarboxylate

2.60 ml (5.13 mmol) of a solution of methylamine (2M) in tetrahydrofuran are added to a solution of 0.380 g (1.03 mmol) of 2-ethoxy-2-oxoethyl 4-(2-(isoquinolin-1-yl)ethyl)-1-piperidinecarboxylate, prepared in stage 7.4., in 10 ml of methanol. Stirring is continued at ambient temperature for 12 hours.

After concentrating under reduced pressure, the residue obtained is purified by chromatography on silica gel, elution being carried out with a 95/5 mixture of dichloromethane and of methanol. A solid is obtained and is recrystallized from a mixture of ethyl acetate and of diisopropyl ether. 0.315 g (0.88 mmol) of product is thus obtained in the form of a white solid.

LC-MS: M+H=356

Melting point (° C.): 126-128

¹H NMR (CDCl₃) δ (ppm): 8.50 (d, 1H), 8.15 (d, 1H), 7.90 (d, 1H), 7.70 (m, 2H), 7.55 (d, 1H), 6.10 (broad s, 1H), 4.60 (broad s, 2H), 4.20 (m, 2H), 3.35 (dd, 2H), 2.90 (m+d, 5H), 1.90 (m, 4H), 1.65 (m, 1H), 1.30 (m, 2H).

The chemical structures and the physical properties of a few compounds according to the invention are illustrated in the following table. In this table:
all the compounds are in the free base form,

n-butyl represents a linear butyl group.

TABLE

| | |
|---|---|
| | (I) |
| 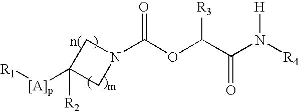 | |

| | | | | | | | | M.p. (° C.) |
|---|---|---|---|---|---|---|---|---|
| No. | R₁ | [A]ₚ | R₂ | n | m | R₃ | R₄ | (M + H) |
| 1. | phenyl | bond | H | 2 | 2 | H | H | 160-162 |
| 2. | phenyl | bond | H | 2 | 2 | H | CH₃ | 76-78 |
| 3. | 3-CF₃-phenyl | bond | H | 2 | 2 | H | H | (331) |
| 4. | 3-CF₃-phenyl | bond | H | 2 | 2 | H | CH₃ | (345) |
| 5. | 5-isobutylpyrid-2-yl | bond | H | 2 | 2 | H | CH₃ | 98-100 |
| 6. | 6-isobutylpyrid-2-yl | bond | H | 2 | 2 | H | CH₃ | (334) |
| 7. | 6-cyclopentylpyrid-2-yl | bond | H | 2 | 2 | H | CH₃ | (346) |
| 8. | 5-(4-F-phenyl)pyrid-2-yl | bond | H | 2 | 2 | H | CH₃ | 151-153 |
| 9. | 6-(4-F-phenyl)pyrid-2-yl | bond | H | 2 | 2 | H | CH₃ | 104-106 |
| 10. | 6-(4-Cl-phenyl)pyrid-2-yl | bond | H | 2 | 2 | H | CH₃ | 136-138 |
| 11. | 5-(4-CF₃-phenyl)pyrid-2-yl | bond | H | 2 | 2 | H | CH₃ | 203-205 |
| 12. | 6-(4-CF₃-phenyl)pyrid-2-yl | bond | H | 2 | 2 | H | CH₃ | 128-130 |
| 13. | 5-(3-CF₃-phenyl)-1- | bond | H | 2 | 2 | H | CH₃ | 160-162 |
| | methylpyrazol-3-yl | | | | | | | |
| 14. | 4-phenylimidazol-1-yl | bond | H | 2 | 2 | H | CH₃ | 192-194 |
| 15. | 5-phenyl-1,3,4-oxadiazol- | bond | H | 2 | 2 | H | H | 152-154 |
| | 2-yl | | | | | | | |
| 16. | 5-phenyl-1,3,4-oxadiazol- | bond | H | 2 | 2 | H | CH₃ | 114-116 |
| | 2-yl | | | | | | | |
| 17. | 5-(4-F-phenyl)-1,3,4-oxa- | bond | H | 2 | 2 | H | H | 158-160 |
| | diazol-2-yl | | | | | | | |
| 18. | 5-(4-F-phenyl)-1,3,4-oxa- | bond | H | 2 | 2 | H | CH₃ | 163-165 |
| | diazol-2-yl | | | | | | | |
| 19. | 5-(3-CF₃-phenyl)- | bond | H | 2 | 2 | H | H | 130-130 |
| | 1,3,4-oxadiazol-2-yl | | | | | | | |
| 20. | 5-(3-CF₃-phenyl)- | bond | H | 2 | 2 | H | CH₃ | 123-125 |
| | 1,3,4-oxadiazol-2-yl | | | | | | | |
| 21. | 3-(3-CF₃-phenyl)- | bond | H | 2 | 2 | H | H | 133-135 |
| | 1,2,4-oxadiazol-5-yl | | | | | | | |
| 22. | 3-(3-CF₃-phenyl)- | bond | H | 2 | 2 | H | CH₃ | 119-121 |
| | 1,2,4-oxadiazol-5-yl | | | | | | | |
| 23. | benzoxazol-2-yl | bond | H | 2 | 2 | H | CH₃ | 137-139 |
| 24. | benzothiazol-2-yl | bond | H | 2 | 2 | H | H | 148-150 |
| 25. | benzothiazol-2-yl | bond | H | 2 | 2 | H | CH₃ | 120-122 |
| 26. | benzimidazol-2-yl | bond | H | 2 | 2 | H | CH₃ | 213-215 |
| 27. | benzimidazol-1-yl | bond | H | 2 | 2 | H | H | 206-208 |
| 28. | 2-phenylbenzimidazol-1-yl | bond | H | 2 | 2 | H | CH₃ | 193-195 |
| 29. | benzotriazol-1-yl | bond | H | 2 | 2 | H | CH₃ | 129-131 |
| 30. | 5-CF₃-benzotriazol-1-yl | bond | H | 2 | 2 | H | H | 152-154 |
| 31. | indol-1-yl | bond | H | 2 | 2 | H | H | 178-180 |
| 32. | 4-Br-phenyl | bond | OH | 2 | 2 | H | CH₃ | 57-60 |
| 33. | 4-(4-F-phenyl)phenyl | bond | OH | 2 | 2 | H | CH₃ | 212-214 |
| 34. | 4-(4-Cl-phenyl)phenyl | bond | OH | 2 | 2 | H | CH₃ | 223-225 |
| 35. | 4-(4-CH₃-phenyl)phenyl | bond | OH | 2 | 2 | H | CH₃ | 179-181 |
| 36. | 4-(4-(n-butyl)phenyl)- | bond | OH | 2 | 2 | H | CH₃ | (425) |
| | phenyl | | | | | | | |
| 37. | 4-(4-CF₃-phenyl)phenyl | bond | OH | 2 | 2 | H | CH₃ | 191-193 |
| 38. | 4-(4-CH₃O-phenyl)phenyl | bond | OH | 2 | 2 | H | CH₃ | 175-176 |
| 39. | 4-(4-C₂H₅O-phenyl)phenyl | bond | OH | 2 | 2 | H | CH₃ | 165-167 |
| 40. | 4-(3-Cl, 4-Cl-phenyl)- | bond | OH | 2 | 2 | H | CH₃ | 156-158 |
| | phenyl | | | | | | | |
| 41. | 4-(3-F, 4-CH₃O-phenyl)- | bond | OH | 2 | 2 | H | CH₃ | (417) |
| | phenyl | | | | | | | |
| 42. | 4-(3-Cl, 4-F-phenyl)phenyl | bond | OH | 2 | 2 | H | CH₃ | 123-125 |
| 43. | naphth-2-yl | CH₂ | H | 2 | 2 | H | CH₃ | 150-152 |
| 44. | 4-phenylphenyl | CH₂ | H | 2 | 2 | H | CH₃ | 115-117 |
| 45. | 6-cyclopentylpyrid-2-yl | CH₂ | H | 2 | 2 | H | CH₃ | (360) |
| 46. | 6-(4-F-phenyl)pyrid-2-yl | CH₂ | H | 2 | 2 | H | CH₃ | 112-114 |
| 47. | indol-1-yl | CH₂ | H | 2 | 2 | H | H | 158-159 |
| 48. | indolin-1-yl | CH₂ | H | 2 | 2 | H | H | 115-116 |
| 49. | 1,2,3,4-tetrahydro- | CH₂ | H | 2 | 2 | H | H | 158-159 |
| | quinolin-1-yl | | | | | | | |
| 50. | 1,2,3,4-tetrahydro- | CH₂ | H | 2 | 2 | H | H | (332) |
| | isoquinolin-2-yl | | | | | | | |
| 51. | pyrrolo[2,3-b]pyrid-1-yl | CH₂ | H | 2 | 2 | H | H | (317) |
| 52. | benzimidazol-1-yl | CH₂ | H | 2 | 2 | H | H | (317) |
| 53. | 4-phenylimidazol-1-yl | CH₂ | H | 2 | 2 | H | H | 124-125 |
| 54. | phenyl | (CH₂)₂ | H | 1 | 2 | H | CH₃ | (291) |
| 55. | 4-F-phenyl | (CH₂)₂ | H | 2 | 2 | H | CH₃ | 150-152 |
| 56. | 3-Cl-phenyl | (CH₂)₂ | H | 2 | 2 | H | CH₃ | 86-88 |
| 57. | 4-Cl-phenyl | (CH₂)₂ | H | 2 | 2 | H | CH₃ | 150-152 |
| 58. | 3-CF₃-phenyl | (CH₂)₂ | H | 2 | 2 | H | CH₃ | 103-105 |
| 59. | 4-CF₃-phenyl | (CH₂)₂ | H | 2 | 2 | H | CH₃ | 131-133 |
| 60. | 3-CN-phenyl | (CH₂)₂ | H | 2 | 2 | H | CH₃ | (330) |
| 61. | 4-CH₃-phenyl | (CH₂)₂ | H | 2 | 2 | H | H | 125-127 |
| 62. | 4-CH₃-phenyl | (CH₂)₂ | H | 2 | 2 | H | CH₃ | 117-119 |
| 63. | 4-CH₃O-phenyl | (CH₂)₂ | H | 2 | 2 | H | H | 123-125 |
| 64. | 4-CH₃O-phenyl | (CH₂)₂ | H | 2 | 2 | H | CH₃ | 122-124 |
| 65. | 2-phenylphenyl | (CH₂)₂ | H | 2 | 2 | H | CH₃ | (381) |
| 66. | 3-phenylphenyl | (CH₂)₂ | H | 2 | 2 | H | CH₃ | 113-115 |
| 67. | naphth-1-yl | (CH₂)₂ | H | 2 | 2 | H | CH₃ | 112-114 |
| 68. | naphth-2-yl | (CH₂)₂ | H | 2 | 2 | H | CH₃ | 106-108 |
| 69. | pyrimidin-2-yl | (CH₂)₂ | H | 2 | 2 | H | CH₃ | 160-170 |
| 70. | pyrimidin-5-yl | (CH₂)₂ | H | 2 | 2 | H | CH₃ | 123-125 |
| 71. | 6-cyclopentylpyrid-2-yl | (CH₂)₂ | H | 2 | 2 | H | CH₃ | (374) |
| 72. | 6-(pyrrolidin-1-yl)- | (CH₂)₂ | H | 2 | 2 | H | CH₃ | 130-132 |
| | pyrid-2-yl | | | | | | | |
| 73. | thiazol-2-yl | (CH₂)₂ | H | 2 | 2 | H | CH₃ | 97-99 |
| 74. | isoquinolin-1-yl | (CH₂)₂ | H | 2 | 2 | H | CH₃ | 126-128 |
| 75. | 1,2,3,4-tetrahydro- | (CH₂)₂ | H | 2 | 2 | H | H | (346) |
| | quinolin-1-yl | | | | | | | |
| 76. | 1,2,3,4-tetrahydro- | (CH₂)₂ | H | 2 | 2 | H | H | 112-114 |
| | isoquinolin-2-yl | | | | | | | |
| 77. | indol-1-yl | (CH₂)₂ | H | 2 | 2 | H | H | (330) |
| 78. | indolin-1-yl | (CH₂)₂ | H | 2 | 2 | H | H | 92-93 |
| 79. | pyrrolo[2,3-b]pyrid-1-yl | (CH₂)₂ | H | 2 | 2 | H | H | (331) |
| 80. | benzimidazol-1-yl | (CH₂)₂ | H | 2 | 2 | H | H | 181-182 |
| 81. | 4-phenylimidazol-1-yl | (CH₂)₂ | H | 2 | 2 | H | H | 183-184 |
| 82. | 3-Cl-phenyl | (CH₂)₃ | H | 2 | 2 | H | CH₃ | 92-94 |
| 83. | 4-Cl-phenyl | (CH₂)₃ | H | 2 | 2 | H | CH₃ | 118-120 |
| 84. | 3-CF₃-phenyl | (CH₂)₃ | H | 2 | 2 | H | CH₃ | 106-108 |
| 85. | 4-CF₃-phenyl | (CH₂)₃ | H | 2 | 2 | H | CH₃ | 111-113 |
| 86. | 3-CN-phenyl | (CH₂)₂ | H | 2 | 2 | H | CH₃ | 118-120 |
| 87. | 2-phenylphenyl | (CH₂)₃ | H | 2 | 2 | H | CH₃ | (395) |
| 88. | 3-phenylphenyl | (CH₂)₃ | H | 2 | 2 | H | CH₃ | 116-118 |
| 89. | naphth-1-yl | (CH₂)₃ | H | 2 | 2 | H | CH₃ | (369) |
| 90. | naphth-2-yl | (CH₂)₃ | H | 2 | 2 | H | CH₃ | 112-114 |
| 91. | pyrimidin-2-yl | (CH₂)₃ | H | 2 | 2 | H | CH₃ | 105-107 |
| 92. | pyrimidin-5-yl | (CH₂)₃ | H | 2 | 2 | H | CH₃ | 105-107 |
| 93. | thiazol-2-yl | (CH₂)₃ | H | 2 | 2 | H | CH₃ | (326) |
| 94. | 3-Cl-phenyl | C═C | H | 2 | 2 | H | CH₃ | 85-87 |
| 95. | 4-Cl-phenyl | C═C | H | 2 | 2 | H | CH₃ | 122-124 |
| 96. | 3-CF₃-phenyl | C═C | H | 2 | 2 | H | CH₃ | (369) |
| 97. | 4-CF₃-phenyl | C═C | H | 2 | 2 | H | CH₃ | 134-136 |
| 98. | 3-CN-phenyl | C═C | H | 2 | 2 | H | CH₃ | (326) |
| 99. | 2-phenylphenyl | C═C | H | 2 | 2 | H | CH₃ | (377) |
| 100. | 3-phenylphenyl | C═C | H | 2 | 2 | H | CH₃ | (377) |
| 101. | naphth-1-yl | C═C | H | 2 | 2 | H | CH₃ | (351) |
| 102. | naphth-2-yl | C═C | H | 2 | 2 | H | CH₃ | (351) |
| 103. | pyrimidin-2-yl | C═C | H | 2 | 2 | H | CH₃ | (303) |
| 104. | pyrimidin-5-yl | C═C | H | 2 | 2 | H | CH₃ | 136-138 |
| 105. | thiazol-2-yl | C═C | H | 2 | 2 | H | CH₃ | (308) |
| 106. | 3-Cl-phenyl | C═CCH₂ | H | 2 | 2 | H | CH₃ | 91-93 |
| 107. | 4-Cl-phenyl | C═CCH₂ | H | 2 | 2 | H | CH₃ | 101-103 |
| 108. | 3-CF₃-phenyl | C═CCH₂ | H | 2 | 2 | H | CH₃ | 113-115 |
| 109. | 4-CF₃-phenyl | C═CCH₂ | H | 2 | 2 | H | CH₃ | 112-114 |
| 110. | 3-CN-phenyl | C═CCH₂ | H | 2 | 2 | H | CH₃ | 112-114 |
| 111. | 2-phenylphenyl | C═CCH₂ | H | 2 | 2 | H | CH₃ | 99-101 |
| 112. | 3-phenylphenyl | C═CCH₂ | H | 2 | 2 | H | CH₃ | (391) |
| 113. | naphth-1-yl | C═CCH₂ | H | 2 | 2 | H | CH₃ | 98-100 |
| 114. | naphth-2-yl | C═CCH₂ | H | 2 | 2 | H | CH₃ | 99-101 |
| 115. | pyrimidin-2-yl | C═CCH₂ | H | 2 | 2 | H | CH₃ | 91-93 |
| 116. | pyrimidin-5-yl | C═CCH₂ | H | 2 | 2 | H | CH₃ | 113-115 |
| 117. | thiazol-2-yl | C═CCH₂ | H | 2 | 2 | H | CH₃ | 112-114 |
| 118. | 6-(pyrrolidin-1-yl)- | CH₂ | H | 2 | 2 | H | CH₃ | 119-121 |
| | pyrid-2-yl | | | | | | | |
| 119. | 6-(1-isopropylpiperidin- | (CH₂)₂ | H | 2 | 2 | H | CH₃ | (431) |
| | 4-yl)pyrid-2-yl | | | | | | | |

The compounds of the invention have formed the subject of pharmacological trials which make it possible to determine their inhibitory effect on the enzyme FAAH (Fatty Acid Amido Hydrolase).

The inhibitory activity was demonstrated in a radioenzymatic test based on the measurement of the product of hydrolysis ((1-³H)ethanolamine) of ((1-³H)ethanolamine)-anandamide by FAAH (Life Sciences (1995), 56, 1999-2005 and Journal of Pharmacology and Experimental Therapeutics (1997), 283, 729-734). Thus, mouse brains (minus the cerebellum) are removed and stored at −80° C. The membrane homogenates are prepared at the time of use by homogenization of the tissues with a Polytron in a 10 mM Tris-HCl buffer (pH 8.0) comprising 150 mM NaCl and 1 mM EDTA. The enzymatic reaction is subsequently carried out in 70 μl of buffer comprising bovine serum albumin free from fatty acids (1 mg/ml). The test compounds, at various concentrations, the ((1-³H)ethanolamine)-anandamide (specific activity of 15-20 Ci/mmol), diluted to 10 μM with non-radiolabelled anandamide, and the membrane preparation (400 μg of frozen tissue per assay) are successively added. After 15 minutes at 25° C., the enzymatic reaction is halted by addition of 140 μl of chloroform/methanol (2:1). The mixture is stirred for 10 minutes and is then centrifuged at 3500 g for 15 minutes. An aliquot (30 μl) of the aqueous phase comprising the (1-³H)ethanolamine is counted by liquid scintillation.

Under these conditions, the most active compounds of the invention exhibit IC₅₀ values (concentration which inhibits the control enzymatic activity of FAAH by 50%) of between 0.001 and 1 μM.

For example, compounds Nos. 39 and 40 in the table exhibit IC₅₀ values of 0.095 and 0.098 μM respectively.

It is therefore apparent that the compounds according to the invention have an inhibitory activity on the enzyme FAAH.

The in vivo activity of the compounds of the invention was evaluated in a test for analgesia.

Thus, the intraperitoneal (i.p.) administration of PBQ (phenylbenzoquinone, 2 mg/kg in a 0.9% sodium chloride solution comprising 5% of ethanol) to male OF1 mice weighing 25 to 30 g causes abdominal tractions, on average 30 twisting or contracting motions during the period from 5 to 15 minutes after injection. The test compounds are administered, orally (p.o.) or intraperitoneally (i.p.) in suspension in 0.5% Tween 80, 60 minutes or 120 minutes before the administration of PBQ. Under these conditions, the most powerful compounds of the invention reduce by 35 to 70% the number of tractions induced by the PBQ, within a range of doses of between 1 and 30 mg/kg.

For example, compound No. 57 in the table reduces by 37% and by 74% the number of tractions induced by the PBQ, at a dose of 3 mg/kg p.o., at 60 minutes and at 120 minutes respectively.

As discussed earlier, the enzyme FAAH (Chemistry and Physics of Lipids, (2000), 108, 107-121) catalyses the hydrolysis of endogenous derivatives of amides and esters of various fatty acids, such as N-arachidonoylethanolamine (anandamide), N-palmitoylethanolamine, N-oleoylethanolamine, oleamide or 2-arachidonoylglycerol. These derivatives have various pharmacological activities by interacting, inter alia, with the cannabinoid and vanilloid receptors.

The compounds of the invention block this decomposition pathway and increase the tissue level of these endogenous substances. They can therefore be used in the prevention and treatment of pathologies in which endogenous cannabinoids and/or any other substrate metabolized by the enzyme FAAH are involved.

The use of a compound of formula (I), in the base or pharmaceutically acceptable salt, hydrate or solvate form, in the preparation of a medicament intended to treat the above-mentioned pathologies forms an integral part of the invention.

Another subject-matter of the invention is medicaments which comprise a compound of formula (I) or a pharmaceutically acceptable salt, hydrate or solvate of the compound of formula (I). These medicaments are used in therapeutics, in particular in the treatment of the above-mentioned pathologies.

According to another of its aspects, the present invention relates to pharmaceutical compositions including, as active principle, at least one compound according to the invention. These pharmaceutical compositions comprise an effective dose of a compound according to the invention or a pharmaceutically acceptable salt, hydrate or solvate of the said compound and optionally one or more pharmaceutically acceptable excipients.

The said excipients are chosen, depending on the pharmaceutical form and the method of administration desired, from the usual excipients which are known to a person skilled in the art.

In the pharmaceutical compositions of the present invention for oral, sublingual, subcutaneous, intramuscular, intravenous, topical, local, intrathecal, intranasal, transdermal, pulmonary, ocular or rectal administration, the active principle of formula (I) above or its optional salt, solvate or hydrate can be administered in a single-dose administration form, as a mixture with conventional pharmaceutical excipients, to animals and to man for the prophylaxis or the treatment of the above disorders or diseases.

Appropriate single-dose administration forms comprise oral forms, such as tablets, soft or hard gelatin capsules, powders, granules, chewing gums and oral solutions or suspensions, forms for sublingual, buccal, intratracheal, intraocular or intranasal administration or for administration by inhalation, forms for subcutaneous, intramuscular or intravenous administration and forms for rectal or vaginal administration. For topical application, the compounds according to the invention can be used in creams, ointments or lotions.

By way of example, a single-dose administration form of a compound according to the invention in the form of a tablet can comprise the following components:

| | | |
|---|---|---|
| Compound according to the invention | 50.0 | mg |
| Mannitol | 223.75 | mg |
| Croscarmellose sodium | 6.0 | mg |
| Maize starch | 15.0 | mg |
| Hydroxypropylmethylcellulose | 2.25 | mg |
| Magnesium stearate | 3.0 | mg |

The said single-dose forms comprise a dose which makes possible a daily administration of 0.01 to 20 mg of active principle per kg of body weight, depending upon the pharmaceutical dosage form.

There may be specific cases where higher or lower dosages are appropriate; such dosages also come within the invention. According to the usual practice, the dosage appropriate to each patient is determined by the doctor according to the method of administration, the weight and the response of the said patient.

According to another of its aspects, the invention also relates to a method for the treatment of the pathologies indicated above which comprises the administration of an effective dose of a compound according to the invention, of one of its pharmaceutically acceptable salts or of a solvate or of a hydrate of the said compound.

What is claimed is:

1. A compound corresponding to the formula (I) 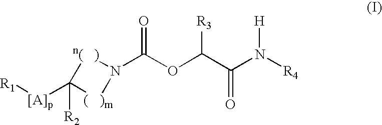
in which
m and n represent integers ranging from 1 to 3 such that m+n is an integer ranging from 2 to 5;
p represents an integer ranging from 1 to 7;
A represents a single bond or is chosen from one or more groups X, Y and/or Z;
X represents a methylene group optionally substituted by one or two C₁₋₆-alkyl, C₃₋₇-cycloalkyl or C₃₋₇-cycloalkyl-C₁₋₃-alkylene groups;
Y represents either a C₂-alkenylene group optionally substituted by one or two C₁₋₆-alkyl, C₃₋₇-cycloalkyl or C₃₋₇-cycloalkyl-C₁₋₃-alkylene groups; or a C₂-alkynylene group;
Z represents a group of formula: 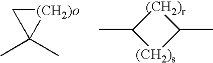
o represents an integer ranging from 1 to 5;
r and s represent integers and are defined such that r+s is a number ranging from 1 to 5;
R₁ represents an R₅ group optionally substituted by one or more R₆ and/or R₇ groups;
R₂ represents a hydrogen or fluorine atom or a hydroxyl, C₁₋₆-alkoxy or NR₈R₉ group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, pyridazinyl, pyrazinyl, triazinyl, pyrrolyl, furyl, thienyl, imidazolyl, oxazolyl, thiazolyl, pyrazolyl, isoxazolyl, isothiazolyl, oxadiazolyl, thiadiazolyl, triazolyl, tetrazolyl, naphthyl, quinolinyl, tetrahydroquinolinyl, isoquinolinyl, tetrahydroisoquinolinyl, quinazolinyl, quinoxalinyl, phthalazinyl, cinnolinyl, naphthyridinyl, benzofuranyl, dihydrobenzofuranyl, benzothienyl, dihydrobenzothienyl, indolyl, indolinyl, indazolyl, isoindolyl, benzimidazolyl, benzoxazolyl, benzisoxazolyl, benzothiazolyl, benzisothiazolyl, benzotriazolyl, benzoxadiazolyl, benzothiadiazolyl, pyrrolopyridyl, furopyridyl, thienopyridyl, imidazopyridyl, oxazolopyridyl, thiazolopyridyl, pyrazolopyridyl, isoxazolopyridyl or isothiazolopyridyl;
R₆ represents a halogen atom or a cyano, nitro, C₁₋₆-alkyl, C₃₋₇-cycloalkyl, C₁₋₆-alkoxy, hydroxyl, C₁₋₆-thioalkyl, C₁₋₆-fluoroalkyl, C₁₋₆-fluoroalkoxy, C₁₋₆-fluorothioalkyl, NR₈R₉, NR₈COR₉, NR₈CO₂R₉, NR₈SO₂R₉, COR₈, CO₂R₈, CONR₉R₉, SO₂R₈, SO₂NR₈R₉ or —O—(C₁₋₃-alkylene)-O— group or a ring chosen from the azetidine, pyrrolidine, piperidine, morpholine, thiomorpholine, azepine or piperazine rings, this ring optionally being substituted by a C₁₋₆-alkyl or benzyl group;
R₇ represents a phenyl, phenyloxy, benzyloxy, naphthyl, pyridyl, pyrimidinyl, pyridazinyl or pyrazinyl group; it being possible for the R₇ group or groups to be substituted by one or more R₆ groups which are identical to or different from one another;
R₈ and R₉ represent, independently of one another, a hydrogen atom or a C₁₋₆-alkyl group;
in the base form or in the form of an addition salt with an acid, of a hydrate or of a solvate.

2. The compound of formula (I) according to claim 1, wherein;
m and n represent integers equal to 1 or 2 such that m+n is an integer ranging from 2 to 4;
p represents an integer ranging from 1 to 3;
A represents a single bond or a methylene or C₂-alkynylene group;
R₁ represents an R₅ group optionally substituted by one or more R₆ and/or R₇ groups;
R₂ represents a hydrogen atom or a hydroxyl group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, imidazolyl, thiazolyl, pyrazolyl, isoxazolyl, oxadiazolyl, naphthyl, tetrahydroquinolinyl, isoquinolinyl, tetrahydroiso-quinolinyl, indolyl, indolinyl, benzimidazolyl, benzoxazolyl, benzothiazolyl, benzotriazolyl or pyrrolopyridyl;
R₆ represents a halogen atom or a cyano, C₁₋₆-alkyl, C₃₋₇-cycloalkyl, C₁₋₆-alkoxy or C₁₋₆-fluoroalkyl group or a pyrrolidine or piperidine ring, this ring optionally being substituted by a C₁₋₆-alkyl group;
R₇ represents a phenyl group which can be substituted by one or more R₆ groups which are identical to or different from one another;
in the base form or in the form of an addition salt with an acid, of a hydrate or of a solvate.

3. The compound of formula (I) as set forth in claim 2, wherein;
m and n represent integers equal to 1 or 2 such that m+n is an integer ranging from 2 to 4;
p represents an integer ranging from 1 to 3;
A represents a single bond or a methylene or C₂-alkynylene group;
R₁ represents an R₅ group optionally substituted by one or more R₆ and/or R₇ groups;
R₂ represents a hydrogen atom or a hydroxyl group;
R₃ represents a hydrogen atom or a C₁₋₆-alkyl group;
R₄ represents a hydrogen atom or a C₁₋₆-alkyl, C₃₋₇-cycloalkyl or C₃₋₇-cycloalkyl-C₁₋₃-alkyl group;
R₅ represents a group chosen from a phenyl, pyridyl, pyrimidinyl, thiazolyl, isoxazolyl, naphthyl or isoquinolinyl;
R₆ represents a halogen atom or a cyano, C₁₋₆-alkyl, C₃₋₇-cycloalkyl, C₁₋₆-alkoxy or C₁₋₆-fluoroalkyl group or a pyrrolidine or piperidine ring, this ring optionally being substituted by a C₁₋₆-alkyl group;
R₇ represents a phenyl group which can be substituted by one or more R₆ groups which are identical to or different from one another.

4. The compound of formula (I) as set forth in claim 3, wherein;
R₃ represents a hydrogen atom;
R₄ represents a hydrogen atom or a C₁₋₆-alkyl group;
And exists in the base form or in the form of an addition salt with an acid, of a hydrate or a solvate.

5. A process for the preparation of a compound of formula (I) as set forth in claim 1 comprising the conversion of the the carbamate-ester of general formula (IV) 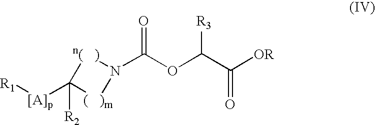
by aminolysis using an amine of general formula R₄NH₂,
in which R₁, A, R₂, R₃, p, m and n are as defined in the formula (I) as set forth in claim 1 and R represents a methyl or ethyl group,
by aminolysis using an amine of general formula R₄NH₂, in which R₄ is as defined in the formula (I) according to claim 1.

6. A compound corresponding to the general formula (IV) 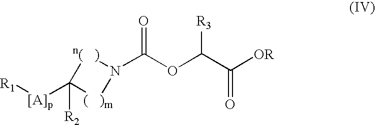
in which R₁, A, R₂, R₃, p, m and n are as defined in the claim 1 and R represents a methyl or ethyl group.

7. A pharmaceutical composition comprising at least one compound of formula (I) as set forth in claim 1 in the base or pharmaceutically acceptable salt, hydrate or solvate form, and optionally one or more pharmaceutically acceptable excipients.

8. A pharmaceutical composition comprising the compound of formula (IV) 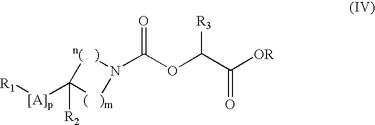
in which R₁, A, R₂, R₃, p, m and n are as defined in claim 1 and R represents a methyl or ethyl group in the base or pharmaceutically acceptable salt, hydrate or solvate form, in combination with one or more pharmaceutically acceptable excipents for use as medicament for the treatment of of a pathology caused by the presence of endogenous cannabinoids and/or any other substrate metabolized by the enzyme fatty acid amido hydrolase (FAAH).

9. A method for the treatment of a pathology caused by the presence of endogenous cannabinoids and/or any other substrate metabolized by the enzyme fatty acid amido hydrolase (FAAH).

10. A method for the treatment of a pathology caused by the presence of endogenous cannabinoids and/or any other substrate metabolized by the enzyme fatty acid amido hydrolase (FAAH) through the administration of the compound as set forth as formula (I) 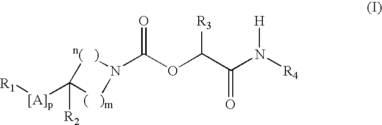
whereas R₁, R₂, R₃, R₄, m, n, and p are as defined in claim 1.

11. A method for the treatment of a pathology caused by the presence of endogenous cannabinoids and/or any other substrate metabolized by the enzyme fatty acid amido hydrolase (FAAH) through the administration of the compound as set forth as formula (IV) 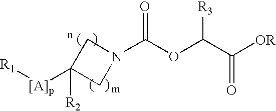
in which R₁, A, R₂, R₃, p, m and n are as defined in the formula (I) according to claim 1 and R represents a methyl or ethyl group.

12. The method of treatment of claim 10 wherein said disease or condition is selected from the group comprising pain, in particular acute or chronic neurogenic pain such as migraine headaches, neuropathic pain, including forms associated with the herpes virus and with diabetes; acute or chronic pain associated with inflammatory diseases such as arthritis, rheumatoid arthritis, osteoarthritis, spondylitis, gout, vasculitis, Crohn's disease, irritable bowel syndrome;
acute or chronic peripheral pain; dizziness, vomiting, nausea, in particular nausea resulting from chemotherapy; eating disorders, in particular anorexia and cachexia of various natures; neurological and psychiatric pathologies, tremors, dyskinesias, dystonias, spasticity, obsessive-compulsive behaviour, Tourette's syndrome, all forms of depression and of anxiety of any nature and origin, mood disorders, psychoses; acute and chronic neurodegenerative diseases: Parkinson's disease, Alzheimer's disease, senile dementia, Huntington's chorea, lesions related to cerebral ischaemia and to cranial and medullary trauma;epilepsy;sleep disorders, including sleep apnoea; cardiovascular diseases, in particular hypertension, cardiac arrhythmias, arteriosclerosis, heart attack, cardiac ischaemia and renal ischaemia; cancers: benign skin tumours, brain tumours and papillomas, prostate tumours, cerebral tumours (glioblastomas, medulloepitheliomas, medulloblastomas, neuroblastomas, tumours of embryonic origin, astrocytomas, astroblastomas, ependymomas, oligodendrogliomas, plexus tumour, neuroepitheliomas, epiphyseal tumour, ependymoblastomas, malignant meningiomas, sarcomatosis, malignant melanomas, schwannomas); disorders of the immune system, in particular autoimmune diseases: psoriasis, lupus erythematosus, diseases of the connective tissue or collagen diseases, Sjogren's syndrome, ankylosing spondylitis, undifferentiated spondylitis, Behcet's disease, cancers: benign skin tumours, brain tumours and papillomas, prostate tumours, cerebral tumours (glioblastomas, medulloepitheliomas, medulloblastomas, neuroblastomas, tumours of embryonic origin, astrocytomas, astroblastomas, ependymomas,
oligodendrogliomas, plexus tumour, neuroepitheliomas, epiphyseal tumour, ependymoblastomas, malignant meningiomas, sarcomatosis, malignant melanomas, schwannomas); disorders of the immune system, in particular autoimmune diseases: psoriasis, lupus erythematosus, diseases of the connective tissue or collagen diseases, Sjögren's syndrome, ankylosing spondylitis, undifferentiated spondylitis, Behcet's disease, autoimmune haemolytic anaemia, multiple sclerosis, amyotrophic lateral sclerosis, amyloidosis, graft rejection, diseases affecting the plasmocytic line; allergic diseases: immediate or delayed hypersensitivity, allergic rhinitis or conjunctivitis, contact dermatitis; parasitic, viral or bacterial infectious diseases: AIDS, meningitis; inflammatory diseases, in particular joint diseases: arthritis, rheumatoid arthritis, osteoarthritis, spondylitis, gout, vasculitis, Crohn's disease, irritable bowel syndrome; osteoporosis; eye conditions: ocular hypertension, glaucoma;
pulmonary conditions: diseases of the respiratory tract, bronchospasm, coughing, asthma, chronic bronchitis, chronic obstruction of the respiratory tract gastrointestinal diseases: irritable bowel syndrome, inflammatory intestinal disorders, ulcers, diarrhoea;urinary incontinence and bladder inflammation.

13. The compound of formula (I) according to claim 6, it's base or pharmaceutically acceptable salt, hydrate or solvate form, in combination with pharmaceutically acceptable excipients for the preparation of a medicament intended to prevent or treat a pathology in which endogenous cannabinoids and/or any other substrate metabolized by the enzyme FAAH are involved.

14. The compound of formula (I) according to claim 6, it's base or pharmaceutically acceptable salt, hydrate or solvate form, in combination with pharmaceutically acceptable excipients for the preparation of a medicament for the prevention or treatment of acute or chronic pain, dizziness, vomiting, nausea, eating disorders, neurological and psychiatric pathologies, acute or chronic neurodegenerative diseases, epilepsy, sleep disorders, cardiovascular diseases, renal ischaemia, cancers, disorders of the immune system, allergic diseases, parasitic, viral or bacterial infectious diseases, inflammatory diseases, osteoporosis, eye conditions, pulmonary conditions, gastrointestinal diseases or urinary incontinence.

15. A process for the preparation of a compound of formula(I) 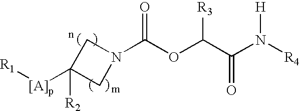
wherein R₁, R₂, R₃, R₄, m, n, and p are as defined in claim 1.
